(19) United States
(12) Patent Application Publication
(10) Pub. No.: US 2024/0194321 A1
(43) Pub. Date: Jun. 13, 2024

(54) METHODS AND SYSTEMS FOR TIMING IMPACT OF NOURISHMENT CONSUMPTION

(71) Applicant: KPN INNOVATIONS, LLC., LAKEWOOD, CO (US)

(72) Inventor: Kenneth Neumann, LAKEWOOD, CO (US)

(73) Assignee: KPN INNOVATIONS, LLC., LAKEWOOD, CO (US)

(21) Appl. No.: 18/410,475

(22) Filed: Jan. 11, 2024

Related U.S. Application Data
(63) Continuation-in-part of application No. 17/592,047, filed on Feb. 3, 2022, now Pat. No. 11,894,124.
Application No. 17/592,047 is a continuation of application No. 17/106,610, filed on Nov. 30, 2020, now Pat. No. 11,270,789.

(51) Int. Cl. G16H 20/60; G06N 20/00

(52) U.S. Cl. CPC G16H 20/60; G06N 20/00

(57) ABSTRACT

A system and method for timing impact of nourishment consumption, the system including a computing device configured to receive training data, train a nutrient machine-learning model using the training data, generate a nutrient profile of the subject utilizing the nutrient machine-learning model, determine, using the nutrient profile, a nourishment consumption program, provide, to the subject, the nourishment consumption program, receive a set of nutrition consumption data of the subject as a function of the nourishment consumption program, and generate an updated nutrient profile as a function of the set of nutrition consumption data and the defined time intervals. The computing device further configured to generate audiovisual notifications based on the nutrient consumption program The computing device further configured to provide an updated consumption pattern of the nourishment consumption program as a function of the set of nutrition consumption data and the updated nutrient profile at defined time intervals.

### CROSS-REFERENCE TO RELATED APPLICATIONS

This application is a continuation-in-part of U.S. Non-provisional application Ser. No. 17/592,047, filed on Feb. 3, 2022, and entitled “METHODS AND SYSTEMS FOR TIMING IMPACT OF NOURISHMENT CONSUMPTION,” which is a continuation of U.S. Non-provisional application Ser. No. 17/106,610, filed on Nov. 30, 2020, and entitled “METHODS AND SYSTEMS FOR TIMING IMPACT OF NOURISHMENT CONSUMPTION,” the entirety of which is incorporated herein by reference.

### FIELD OF THE INVENTION

The present invention generally relates to the field of nutrient timing. In particular, the present invention is directed to methods and systems for timing impact of nourishment consumption.

### BACKGROUND

The detection of the concentration level of metabolites, nutrients, and other analytes in individuals may be vitally important to their health. For example, the monitoring of glucose levels is particularly important to individuals with diabetes or pre-diabetes. People with various conditions may need to monitor nutrient levels to determine when, for instance, medication is needed.

### SUMMARY OF THE DISCLOSURE

In an aspect, a system for timing impact of nourishment consumption, the system including a computing device configured to receive training data comprising physiological data, correlated to current nutrient levels of a subject. The computing device further configured to train a nutrient machine-learning model using the training data. The computing device further configured to generate a nutrient profile of the subject utilizing the nutrient machine-learning model. The computing device further configured to determine, using the nutrient profile, a nourishment consumption program. The computing device further configured to provide, to the subject, the nourishment consumption program, receive a set of nutrition consumption data of the subject as a function of the nourishment consumption program, and generate an updated nutrient profile as a function of the set of nutrition consumption data and the defined time intervals. The computing device further configured to generate one or more audiovisual notifications based on the nutrient consumption program, wherein the one or more audiovisual notifications includes a notification of encouragement. The computing device further configured to provide, to the subject, an updated consumption pattern of the nourishment consumption program as a function of the set of nutrition consumption data and the updated nutrient profile at each defined time interval.

In another aspect, a method for timing impact of nourishment consumption, the method including receiving, by a computing device, training data comprising physiological data, correlated to current nutrient levels of a subject. The method further including training, by the computing device, a nutrient machine-learning model using the training data. The method further including generating, by the computing device, a nutrient profile of the subject utilizing the nutrient machine-learning model. The method further including determining, by the computing device, using the nutrient profile, a nourishment consumption program. The method further including providing, by the computing device, to the subject, the nourishment consumption program. The method further including receiving, by the computing device, a set of nutrition consumption data of the subject as a function of the nourishment consumption program. The method further including generating, by the computing device, an updated nutrient profile as a function of the set of nutrition consumption data and the defined time intervals. The method further including generating, by the computing device, one or more audiovisual notifications based on the nutrient consumption program, wherein the one or more audiovisual notifications includes a notification of encouragement. The method further including providing, by the computing device, to the subject, an updated consumption pattern of the nourishment consumption program as a function of the set of nutrition consumption data and the updated nutrient profile at each defined time interval.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating a system for timing impact of nourishment consumption;

FIG. 2 is a diagrammatic representation of a nourishment consumption program;

FIG. 3 is a diagrammatic representation of nourishment consumption program on a user device;

FIG. 4 is a diagrammatic representation of a nutrient profile;

FIG. 5 is a block diagram illustrating an exemplary embodiment of a machine-learning module;

FIG. 6 is a block diagram illustrating an exemplary embodiment of a nourishment program database;

FIG. 7 is a flow diagram illustrating an exemplary workflow of a method for timing impact of nourishment consumption; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for timing impact of nourishment consumption. In an embodiment, the system includes a computing device configured to receive physiological data pertaining to a subject and receive a nutrient profile. In an embodiment, computing device may determine nutrient profile data by training a machine-learning model with physiological data. Nutrient profile may include per-subject pharmacokinetics, or metabolism, absorption, distribution, and excretion rates, for a variety of nutrients. Computing device is configured to determine a nourishment consumption program to time consumption based on the nutrient profile. In an embodiment, computing device may provide compatible alimentary elements linked to a scheduling application and use reacting computing to update nutrient profile and consumption timing at defined intervals.

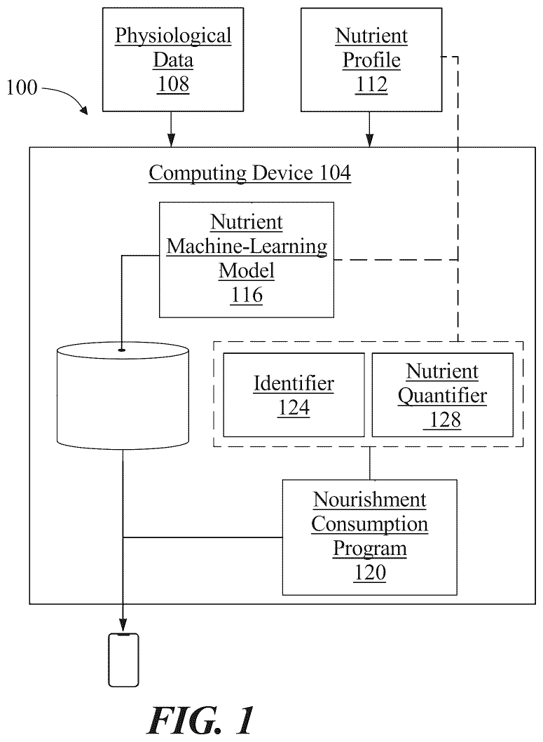

Referring now to FIG. 1, an exemplary embodiment of a system 100 for timing impact of nourishment consumption is illustrated. System includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

With continued reference to FIG. 1, computing device may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 is configured to receive physiological data of a subject. A “physiological data,” as used in this disclosure, is chemical data, data originating from a biological extraction, medical data, and the like. A biological extraction may include data originating from a physical sample, such as a blood panel, lipid panel, metabolic test, genome sequencing, and the like. Physiological data 108 may include genetic data including the presence of single nucleotide polymorphisms (SNPs), mutations, allele designations (dominant, recessive, +/−, etc.), genetic sequencing data, and the like; epigenetic data including methylation patterns, gene expression patterns, enzyme concentrations, specific activity, circulating RNAs, and the like; microbiome data including gut microbiota, ‘good’ flora, transient flora, opportunistic pathogens, bacteria, viruses, parasites, fungi, circulating peptides, biologics, and the like; previous medical history including surgeries, treatments, prescriptions, current and past medications, allergies, family history of disease, diagnoses, prognoses, and the like; physiological data including systolic and diastolic blood pressure, resting heart rate, VO2 max, oxygen saturation, blood cell counts, hemoglobin/hematocrit levels, blood iron concentration, body mass index (BMI), blood sugar, HDL/LDL cholesterol levels, hormone levels, and the like; among any other data that one skilled in the art may recognize as physiological data 108 data. Physiological data 108 may include a variety of data, from a variety of sources, with the data originating from the subject and/or a plurality of subjects, and from a variety of categories and sources, for instance and without limitation, as described in U.S. Nonprovisional application Ser. No. 16/886,647, filed on May 28, 2020, and entitled “METHODS AND SYSTEMS FOR DETERMINING A PLURALITY OF BIOLOGICAL OUTCOMES USING A PLURALITY OF DIMENSIONS OF PHYSIOLOGICAL DATA USER DATA AND ARTIFICIAL INTELLIGENCE,” the entirety of which is incorporated herein by reference.

Continuing in reference to FIG. 1, physiological data 108 data may correspond to a nutritional need of a subject. A “nutritional need,” as used in this disclosure, is a quantity of at least a nutrient and/or of a plurality of nutrients for a subject. Nutrient need may be recommended for subject for maintenance of health, improvement of physiology, addressing a symptom, disease, illness, injury, or any type of malady. Nutrient need may refer to, without limitation, macronutrients, such as protein, including non-essential amino acids, essential amino acids, fats including non-essential fats, essential fats such as long-chain polyunsaturated fatty acids (LC-PUFAs), short-chain polyunsaturated fatty acids (SC-PUFAs), omega fatty acids, carbohydrates, including digestible and non-digestible carbohydrates such as dietary fiber, inulin, psyllium, and methylcellulose; micronutrients, such as vitamin A, thiamin (vitamin B1), riboflavin (vitamin B2), niacin (vitamin B3), pantothenic acid (vitamin B5), vitamin B6, biotin (vitamin B7), folate (vitamin B12), vitamin C, vitamin D2, vitamin D3, vitamin E, vitamin K1, vitamin K2; minerals such as calcium, phosphorous, potassium, sodium, magnesium; trace elements such as iron, sulfur, manganese, selenium, chromium, molybdenum, copper, cobalt; halides such a chloride and iodine; electrolytes and salts including bicarbonate, creatine, and phosphocreatine; caloric content, or any other substance that provides nourishment essential for growth and maintenance of subject.

Continuing in reference to FIG. 1, computing device 104 may receive a nutrient profile of the subject, wherein the nutrient profile includes physiological data 108 data mapped to current nutrient levels of the subject. A “nutrient profile,” as used in this disclosure, is a profile including any nutrient data corresponding to a subject's current nutrient levels and recommended nutrient levels. In some embodiments, nutrient profile may include meal data, meat consumption, salt consumption, caffeine intake, and the like. Nutrient profile 112 may include subject current nutrient levels as they relate to recommended nutrient levels, for instance as numerical values the indicate the amounts relative to one another. Nutrient levels may correspond to blood serum levels of nutrients of current nutrition, for instance as determined from a physiological data. Nutrient levels may correspond to percent daily recommended values (or recommended values determined on a customized, per-subject basis). A nutrient profile 112 may include qualitative values such as “deficiency”, “surplus”, “yes”, “no”, etc., of nutrient levels. A nutrient profile 112 may include quantitative values of nutrient levels such as a numerical values, functions of values, matrices, arrays, vectors, systems of equations, variables, coefficients, metrics, parameters, and the like. A nutrient profile 112 may serve as a “survey” of the current state of nutrition of a subject, including any acute and chronic nutritional deficiencies, nutritional surpluses, recommended and/or calculated nutritional targets, etc.

With continued reference to FIG. 1, in some embodiments, computing device 104 may be configured to receive user input data. “User input data,” for the purposes of this disclosure, is data concerning a user that is input by the user or on behalf of the user. User input data may include exercise data, climate data, meal data, travel data, and the like. In some embodiments, user input data may form part of physiological data 108 and/or nutrient profile 112. In some embodiments, user input data may be input into one or more machine learning processes disclosed below.

Continuing in reference to FIG. 1, receiving nutrient profile 112 may include generating a nutrient machine-learning model, using a machine-learning process, wherein the nutrient machine-learning model is trained with training data that includes a plurality of data entries wherein each entry correlates physiological data 108 data to current nutrient levels of the subject. A “machine learning process,” as used in this disclosure, is a process that automatedly uses a body of data known as “training data” and/or a “training set” to generate an algorithm that will be performed by a computing device/module to produce outputs given data provided as inputs; this is in contrast to a non-machine learning software program where the commands to be executed are determined in advance by a subject and written in a programming language. A nutrient machine-learning model 116 may be generated by training a machine-learning process, algorithm, and/or method, with training data, as described in further detail below.

Continuing in reference to FIG. 1, “training data,” as used herein, is data containing correlations that a machine learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine learning processes as described in further detail below.

Continuing in reference to FIG. 1, training data may include physiological data 108. Training data may originate from the subject, for instance via a questionnaire and a user interface with computing device 104, providing medical history data, retrieving whole genome sequencing, and the like. Training data may be recorded and transmitted to computing device 104 via a wearable device such as a pedometer, gyrometer, accelerometer, motion tracking device, bioimpedance device, ECG/EKG/EEG data, physiological sensors, blood pressure monitor, blood sugar and volatile organic compound (VOC) monitor, and the like. Training data may originate from an individual other than subject, including for instance a physician, lab technician, nurse, caretaker, psychologist, therapist, and the like. Training data may include biomarkers associated with nutrient amounts and quality of subject nutrition, such as red blood cell (RBC) count. RBC count may be elevated due to dehydration, high testosterone. RBC count may be low due to nutrient deficiencies (iron, vitamin B6, vitamin B12, folate), kidney dysfunction, chronic inflammation, anemia, blood loss, and the like. Training data may include hemoglobin levels, which may be elevated due to dehydration, elevated testosterone, poor oxygen deliverability, thiamin deficiency, insulin resistance. Hemoglobin levels may be deceased due to anemia, liver disease, hypothyroidism, exercise, arginine deficiency, protein deficiency, inflammation nutrient deficiencies (vitamin E, magnesium, zinc, copper, selenium, vitamin B6, vitamin A). Training data may include hematocrit levels which may be elevated due to dehydration, elevated testosterone, poor oxygen deliverability, thiamin deficiency, insulin resistance. Hematocrit levels may be deceased due to anemia, liver disease, hypothyroidism, exercise, arginine deficiency, protein deficiency, inflammation nutrient deficiencies (vitamin E, magnesium, zinc, copper, selenium, vitamin B6, vitamin A). Training data may include mean corpuscular hemoglobin (MCH), or a measure of the average weight of hemoglobin per red blood cell. MCH may be elevated (“macrocytic”) due to nutrient deficiencies (vitamin B12, folate, vitamin C), alcohol consumption, thiamin deficiency, and (falsely increased) by hyperlipidemia. MCH may be decreased (“microcytic”) due to iron deficiency, nutrient deficiencies (vitamin B6, copper, zinc, vitamin A, vitamin C). Training data may include measures of the average concentration of hemoglobin in red blood cells, which may be elevated (“macrocytic”) due to nutrient deficiencies (vitamin B12, folate, vitamin C), alcohol consumption, thiamin deficiency, and (falsely increased) by hyperlipidemia. Concentration of hemoglobin may be decreased (“microcytic”) due to iron deficiency, nutrient deficiencies (vitamin B6, copper, zinc, vitamin A, vitamin C). Training data may include data on platelets or small, a nucleated cell fragments in blood that are involved in clotting and important for vascular integrity. Platelets may be increased due to iron deficiency anemia, collagen diseases, hemolytic anemia, blood loss, stress, infection, inflammation. Platelets may be decreased due to alcoholism, liver dysfunction, viral/bacterial infections, pernicious anemia, bleeding. Training data may include cellular dimension assessment, such as measures of the average size of platelets, reflecting their function. Platelets counts may be elevated due to increased platelet production, which is often caused by loss or destruction of existing platelets. Elevated mean platelet volume (MPV) may be associated with vascular disease and mortality, certain cancers, type 2 diabetes, and Hashimoto's thyroiditis. MPV may be decreased due to conditions associated with under-production of platelets such as aplastic anemia or cytotoxic drug therapy. Training data may include red blood cell distribution width, a measurement of the variation in red blood cell size. Typically increased due to nutrient deficiency-related anemias (iron, vitamin A, copper, zinc, vitamin B6). Persons skilled in the art, upon review of this disclosure in its entirety, the range of physiological data that may serve as training data to determine nutrient levels of a subject.

Continuing in reference to FIG. 1, training data may include nutritional input data. A “nutritional input,” as used in this disclosure, is any nutritional value, nutrient amount, or the like, consumed by the subject. A nutritional input may include any alimentary elements consumed by subject, over any designated period of time. An “alimentary element,” as used in this disclosure, is any edible element intended to provide some nutrient value, including hydration, electrolytes, macronutrient, micronutrients, bioactive ingredients, and the like. An alimentary element may include a meal, food item, beverage, supplement, among other items. As a non-limiting example, alimentary element may include an amount of water or an amount of hydration. Persons skilled in the art, upon review of this disclosure in its entirety, the range of nutritional input data, provided by subject or otherwise, that may serve as training data, or input data, in determining nutrient levels of a subject.

Continuing in reference to FIG. 1, computing device 104 may determine the nutrient profile 112 as a function of the nutrient machine-learning model 116. For instance and without limitation, training data may include subject nutritional input (and associated times of consumption) as training data to train nutrient machine-learning model to ‘learn’, based on the subject's physiological data (age, sex, height, weight, lean body mass, BMI, activity level, basal metabolism, food intolerances, digestive issues, metabolic disorders, etc.), the effect alimentary elements have on nutrient profile 112. Nutrient machine-learning model 116 may identify patterns in the training data the relate to, for instance and without limitation, numerical values that describe current nutrient profile categories.

Continuing in reference to FIG. 1, in non-limiting illustrative examples, training data may include physiological data 108 used to train nutrient machine-learning model 116 to derive pharmacokinetics, or per-subject metabolism, absorption, distribution, and excretion rates, for a variety of nutrients and/or alimentary elements. For instance, training data may include blood concentrations (mg/dL) of nutrients (arginine, glucose, iron, etc.) after meal consumption. Persons skilled in the art may appreciate that training a nutrient machine-learning model with such data, over sufficiently great number of training epochs, for a variety of individual nutrients (or foods) for a variety of alimentary element categories (grains, meats, vegetables, fruits, etc.), may result in rates at which each nutrient may increase/decrease after consuming a meal. In this way, training data may be used to determine a nutrient profile 112 that encompasses per-subject kinetics of nutrient metabolism, absorption, and the like, that may be used to inform consumption timing.

Continuing in reference to FIG. 1, for instance and without limitation, training data may include blood test results from blood draws by a primary physician, or blood analyte results from a wearable device, physiological sensor, or the like. Training data may include nutrient levels for blood analytes, such as vitamin A, glucose, magnesium, and calcium. This data may be used to train the nutrient machine-learning model 116 to derive expected nutrient levels and rates of change of the nutrients (vitamin A, glucose, magnesium, and calcium), provided an input of a consumed alimentary element. In this way, nutrient profile 112 may indicate a numerical value relating to the current nutrient level of ‘vitamin A, glucose, magnesium, and calcium’ in a subject after consuming ‘breakfast’, wherein breakfast included ‘apple cinnamon oatmeal’ and ‘whole milk’. The output may include a data structure (nutrient profile 112) that may inform the timing of meals, for instance, based on the ‘current nutrient level’, the ‘target nutrient level’, and ‘nutrient adsorption rates’.

Continuing in reference to FIG. 1, nutrient profile 112 data may be determined at regular time points and extrapolated for diets. In non-limiting illustrative examples, measuring metabolism and/or absorption rates may be performed using a guiding rubric, for example by learning ‘how a ketogenic meal affects blood nutrient levels at 1 hour post-meal, 6 hours post-meal, 24 hours post-meal, etc.’. Alternatively or additionally, training data elements may be collected/recorded and organized for individual alimentary elements, for instance a fruit, vegetable, and the like. In such an example, inputs may include nutrition facts for an alimentary element, and from the relationships identified (mathematically defined in the model), the expected nutrient level in the subject after consumption can be output. Using this output from nutrient machine-learning model 116, system 100 may determine, based on how these nutrient amounts change over time, ‘when’ to plan the next meal.

Continuing in reference to FIG. 1, computing device 104 is configured to determine, using the nutrient profile 112, a nourishment consumption program, wherein the nourishment consumption program includes at least an alimentary element and a time of day for consuming the alimentary element. A “nourishment consumption program,” as used in this disclosure, is a plan that guides the timing of consumption of a subject and an identity of an item for consumption. Nourishment consumption program 120 may include a time of day for consuming an alimentary element, such as a compatible alimentary element. Nourishment consumption program 120 may include timing the consumption of alimentary elements according to a threshold value of a nutrient. For instance and without limitation, nourishment consumption program 120 may include the timing of meals to keep blood glucose below an upper threshold value, and above a lower threshold value. In such an example, nourishment consumption program 120 may include “when” subject should consume a meal to keep blood sugar within the range, wherein range may include a numerical value range. The timing of consumption may then change depending on the alimentary element considered. For instance, ‘times’ indicated in the program may be modified as a function of what is consumed. Meals heavy in simple sugars (monosaccharides/disaccharides) may prompt the next meal to follow closely (+1-3 hours); however, if the carbohydrate profile of a meal includes large quantities of complex sugars (starch/fiber), blood sugar may be within range for up to 4-5 hours afterward. Persons skilled in the art may appreciate that determining timing of consumption may be performed with a respect to variety of goals, or targets, diets, etc. For instance, timing can be optimized to achieve a particular amount of macromolecular nutrients (carbohydrates, fats, proteins), calories, micronutrients (iron, calcium, magnesium), to maintain a certain amount bioactive ingredient, to improve/optimize protein synthesis in muscle tissue for recovery from exercise, adherence to a state of ketosis, and the like.

Continuing in reference to FIG. 1, determining the nourishment consumption program 120 may include retrieving an alimentary element program comprising compatible alimentary elements. An “alimentary element program,” is a collection of alimentary elements provided to subject. An alimentary element program may include compatible alimentary elements. A “compatible alimentary element,” as used in this disclosure, is an alimentary element proscribed to a subject based on the subject's physiological data 108. An alimentary element program may include meals, recipes, grocery items, menu items, supplements, bioactive ingredients, beverages, and the like, that are intended for a subject based on determinations made from physiological data 108 data, such as food allergies and intolerances, improving physiological state of health, decreasing inflammation, addressing chronic nutrient deficiencies, and the like.

Continuing in reference to FIG. 1, compatible alimentary element may include alimentary elements intended to address a nutrition deficiency, reduce inflammation, improve recovery from exercise, improve overall health, among other targeted effects. A compatible alimentary element may include alimentary elements provided as a function of an individual's allergies, food intolerances, philosophical, religions, and lifestyle considerations, among other factors involved. Compatible alimentary element may be generated and provided to a user as a function of a physiological data, such as blood chemistry results, including enzyme concentrations and specific activities for instance of fibrinogen, ferritin, serum amyloid A, α-1-acid glycoprotein, ceruloplasmin, hepcidin, haptoglobin, tumor necrosis factor-α (TNF-α), among other acute phase proteins; for instance cytokine identities and concentrations for instance interleukin-6 (IL-6); blood metabolites identities and concentrations such as blood sugar, LDL and HDL cholesterol content; hormone identities and concentrations such as insulin, androgens, cortisol, thyroid hormones, and the like; erythrocyte sedimentation rate, blood cell counts, plasma viscosity, and other biochemical, biophysical, and physiological properties regarding blood panels, blood tests, and the like, as it relates to biomarkers of inflammation. Compatible alimentary elements may be recommended to a user as a function of these biochemical data with the intention of modifying the biochemical data, for instance by modulating blood sugar, decreasing LDL cholesterol levels, reducing pro-inflammatory biomarkers, minimizing free radicals and oxidative damage, among other targeted effects of alimentary elements on physiological data. For instance, biomarkers of inflammation may include biochemical properties specific to a user such as the level of inflammation as evidence by the presence and concentration of inflammatory biomarkers, post-translational modification of proteins, epigenetic markers, etc., and alimentary elements may be identified and provided to a subject to focus on reducing inflammation for instance and without limitation, as described in U.S. Nonprovisional application Ser. No. 17/007,251 filed Aug. 31, 2020 titled “METHOD AND SYSTEM FOR REVERSING INFLAMMATION IN A USER,” the entirety of which is incorporated herein by reference. The level of inflammation, or any biochemical ailment and/or property of a subject may be enumerated, and based on the numerical value, an alimentary element may be recommended to the subject, from which a nourishment consumption program may be defined. Alternatively or additionally, determining nourishment consumption program that improves the user's health state based on the user's biochemistry may be performed, for instance and without limitation, as described in U.S. Nonprovisional application Ser. No. 16/375,303 filed Apr. 4, 2020 titled “SYSTEMS AND METHODS FOR GENERATING ALIMENTARY INSTRUCTION SETS BASED ON VIBRANT CONSTITUTION GUIDANCE,” the entirety of which is incorporated herein by reference.

Continuing in reference to FIG. 1, determining the nourishment consumption program 120 may include identifying a compatible alimentary element to address a datum of the nutrient profile 112. Nutrient profile 112 may include a variety of data based on physiological data 108, as described above, such as current nutrient levels, rates of metabolism, adsorption, and nutrient threshold values. A “nutrient threshold,” as used in this disclosure, is a numerical value of a nutrient. In non-limiting illustrative examples, nutrient profile 112 may include current levels of water-soluble vitamins, fat-soluble vitamins, minerals, trace elements, blood sugar, cholesterol, lipids, amino acids, phosphocreatine, calories, ATP, rates of extracting each of these nutrients from alimentary elements, and the maximal and minimal nutrient thresholds the subject should maintain. With such data, computing device 104 may identify compatible alimentary elements that may bring subject within the nutrient threshold values and may determine the times of day to initiate consumption of alimentary elements to stay within nutrient thresholds ranges throughout the day-night cycle.

Continuing in reference to FIG. 1, nourishment consumption program 120 may be used to identify a compatible alimentary element to, for instance, address a nutrient deficiency. In non-limiting illustrative examples, computing device 104 may compare nutrient levels in nutrient profile 112 to recommended daily allowance indicated by alimentary element program, and using a mathematical operation such as subtraction, determine if a nutrient deficiency exists. Computing device 104 may match a compatible alimentary element to address the deficiency. Applying the nutrient quantity of an alimentary element may result in a ‘nutrient surplus’ in which the timing of a subsequent meal may be extended, or a different alimentary element selected altogether. Applying the nutrient quantity may indicate a ‘nutrient deficiency’ in which case a second alimentary element may be selected to increase the nutrient amount. The quantity may indicate all nutrients are within threshold numerical value ranges, in which case the subject may not need to consume anything for a time.

Continuing in reference to FIG. 1, computing device may calculate a change in the nutrient profile 112 as a function of timing the compatible alimentary element. Computing device 104 may accept an input of a starting value in nutrient profile 112 and a second input of the nutrition facts data of a compatible alimentary element. Computing device 104 may then use the trained nutrient machine-learning model 116 to generate an output of an updated nutrient profile 112. The model may contain relationships regarding the pharmacokinetics of nutrient absorption of macromolecules, micronutrients, etc. The updated nutrient profile 112, which may reflect changes in nutrient levels after consumption, may show nutrients that were obtained, not obtained, an/or depleted. The resulting updated nutrient profile 112 data from using the trained model and the inputs may then inform nourishment consumption program 120, re-calculating the timing of the next meal as nutrient levels change. Persons skilled in the art may appreciate that system 100 may iteratively update nutrient profile 112 at defined intervals, for instance (5 times daily; once an hour, after a meal is eaten), to inform meal timing.

Continuing in reference to FIG. 1, calculating a change in the nutrient profile 112 as a function of timing the compatible alimentary element may include calculating differences in individual nutrient levels in subject. For instance, nutrient profile 112 may include essential amino acid levels for subject as a function of how much protein they have consumed and the protein sources. Computing device 104 may quantify changes in essential amino acid levels after lunch, and use that calculation to best time, to achieve daily recommended amino acid levels, especially for branch chain amino acids (BCAAs). Computing device 104 may select alimentary elements for timing use a mathematical operation, such as addition or subtraction, for instance by adding the amount of protein per serving to the current nutrient levels. Computing device 104 may optimize timing by using a system of equations and/or mathematical expressions to calculate rates (or velocity) of change in the nutrient levels as a function of time. In such an example, the first derivative may be the velocity of reaction (metabolism), second derivative is acceleration (nutrient absorption), and third derivative is the ‘jerk’, or rate of change of acceleration. The third derivative may refer to ‘how long nutrients will continue to increase prior to needing next meal’ after eating. Computing device 104 may employ a variety of methods to calculate a change based on relationships identified by nutrient machine-learning model 116. For instance, a multi-variable system of equations, a matrix, vector analysis, series of functions, transforms, derivatives, and the like, may be discovered by nutrient machine-learning model 116 for mapping how nutrient levels change over time, or react to eating a meal, with sufficient physiological data.

Continuing in reference to FIG. 1, calculating a change in the nutrient profile 112 as a function of timing the compatible alimentary element may include calculating differences in individual nutrient levels in subject Timing may be affected by physiological data regarding fitness data, for instance from a fitness tracking application, wearable device, etc.

Continuing in reference to FIG. 1, nourishment consumption program 120 may include a queue of a plurality of compatible alimentary elements, wherein each compatible alimentary element includes an identifier. An “identifier,” as used in this disclosure, is a datum of identifiable information relating to a compatible alimentary element. An identifier 124 may include alimentary element name, serving size, price, distributor, restaurant identity, nutrition facts, among other identifiable information for an alimentary element. An identifier 124 may include information necessary for ordering compatible alimentary element, for instance via a mobile application, a web browser, and the Internet etc. An identifier 124 may include information detailing the identity of a compatible alimentary element and perhaps why it is necessary for the subject, how it may improve health, etc. A “queue,” as used in this disclosure, is a collection of elements that are maintained in a sequence and can be modified by the addition of entities and removal of elements from the sequence. In non-limiting illustrative examples, the queue may have an “active end” and a “reserve end,” wherein the active end is the ‘most appropriate compatible alimentary element and time of consuming’ to be displayed such as by timing, or some other discriminating criteria; additionally, there may be related alimentary elements that are in the queue “behind” the first active end alimentary element and alternatives nearer the reserve end. In further non-limiting illustrative examples, a user may indicate via a graphical user interface that they do not want an alimentary element, whereby computing device 104 may remove it from the active end and push up by one place the next alimentary elements in the queue. In such an example, computing device 104 may add a newly generated alimentary element to the reserve end to maintain a list that a user may view, scroll through, select/deselect, or the like.

Continuing in reference to FIG. 1, nourishment consumption program 120 may include a time associated with the identifier. A “time,” as used in this disclosure, is a datum of chronologically identifiable data used for communicating the timing of consumption. The time may include a time of day, a countdown, or elapsed time for the next alimentary element. Time may include a dynamic time counter, which visibly changes as a function of nutritional input; alternatively or additionally, time may include predetermined times, such as a schedule linked to a calendar. In non-limiting illustrative examples, since each compatible alimentary element may impose a different effect on nutrient profile 112, the identity of each may include a unique time table for consumption.

Continuing in reference to FIG. 1, nourishment consumption program 120 may include a nutrient quantifier for adjusting the nutrient profile 112 as a function of consumption of an alimentary element associated with the identifier. A “nutrient quantifier,” as used in this disclosure, is a metric, or instruction, that includes an effect on nutrition for an alimentary element. A nutrient quantifier 128 may include numerical values, for instance, as to which nutrients should decrease after consuming. A nutrient quantifier may include a series of values as a function, for instance, which details how a series of nutrient levels change over time after consuming a particular alimentary element, such as blood sugar and sodium change over a 6 hour period from consuming a cheeseburger. A nutrient quantifier 128 may include an instruction, or logical rule, which dictates how the nutrient profile 112 should be modified from each alimentary element consumed, including how to change the time (to next meal), and which alimentary element identifiers should be added/removed (from queue). Nutrient quantifier 128 may include, in non-limiting illustrative examples, that eating a particular snack +1 hour after consuming a first meal may extend the timing for a second meal for another 3 hours, and perhaps change the identity of the second meal, depending on what the snack provides.

Continuing in reference to FIG. 1, nourishment consumption program 120 may modify identifier 124, time, and nutrient quantifier 128 based on evolutionary considerations such as circadian rhythm, among other considerations. Circadian rhythms are self-sustained approximately 24-hour oscillations in behavior, physiology, and metabolism. These rhythms have evolved to permit organisms to effectively respond to the predictable daily change in the light: dark cycle and the resultant rhythms in food availability encountered in nature. Genetic, epigenetic, biochemical, and physiological studies have revealed more than 10% of expressed genes in any organ exhibit circadian oscillation, and this is seen in liver metabolism, musculoskeletal tissue metabolism, appetite control, blood panel results, etc. These rhythmic transcripts encode key rate-determining steps in neuroendocrine, signaling, and metabolic pathways. Such regulation temporally separates cellular processes and optimizes cellular and organismal fitness. Although the circadian clock is cell-autonomous and has been identified in the majority of tissue types, the circadian system is organized in a hierarchical manner in which the hypothalamic suprachiasmatic nucleus (SCN) of the hypothalamus functions as the master circadian clock (also regulating appetite control) that uses both diffusible and synaptic mechanisms to orchestrate circadian rhythms in the peripheral organs at appropriate phase. For instance, photoreceptive retinal ganglion cells (light harvesting system) send ambient light information to the SCN through monosynaptic connection to ensure that the circadian system is entrained to the daily light: dark cycle. This circadian que, among numerous others, may be reflected in a physiological data 108, for system 100 to accurately determine per-subject circadian rhythm. Nutrient profile 112 may include circadian rhythm dietary patterns, eating timing, sleep cycles, etc. For instance, nutritional input data of subject may be used as an input to determine nourishment consumption program 120 timing, wherein patterns in nutritional input (when a subject eats) may help identify circadian rhythm consumption patterns. Such consumption patterns, reflected in nutrient profile 112, may assist in determining personalized, highly accurate times of day for optimized consumption. Nourishment consumption program 120 may include identifier 124, time, and nutrient quantifier 128, that is based on a circadian rhythm as detailed from nutrient profile 112 of a subject; nutrient machine-learning model 116 may identify and describe relationships in training data that capture per-subject circadian rhythm model of nutrition.

Continuing in reference to FIG. 1, nourishment consumption program 120 may include identifier 124, time, and nutrient quantifier 128 based on cultural considerations such as the standard 3-meal day, diet types and dieting fads, among other considerations. Typical breakfast, lunch, and dinner meals may be difficult to distinguish because skipping meals, snacking, and erratic eating patterns have become more prevalent. Such eating styles may have various effects on cardiometabolic health markers, namely obesity, lipid profile, insulin resistance, blood pressure, heart rate, VO₂ max, etc. Nourishment consumption program 120 may include nutrient quantifiers 128 and times aimed at various consumption models such as ‘skipping breakfast’, ‘intermittent fasting’, ‘decreasing meal frequency’ (number of daily eating occasions) and modify timing of consumption based on these paradigms. Consumption patterns may be detailed in nutrient profile 112 or may be collected as inputs via a subject interaction with a user device, such as via a questionnaire provided by a graphical user interface. Furthermore, nourishment consumption program 120 may include program definitions/instructions for meals, snacks, and consumption for use in identifying per-subject consumption patterns to refine nourishment timing more accurately.

Continuing in reference to FIG. 1, providing the nourishment consumption program 120 may include generating, via a graphical user interface, a representation of the nourishment consumption program 120. A “graphical user interface,” as used in this disclosure, is any form of a user interface that allows a subject to interface with an electronic device through graphical icons, audio indicators, text-based interface, typed command labels, text navigation, and the like, wherein the interface is configured to provide information to the subject and accept input from the subject. Graphical user interface may accept subject input, wherein subject input may include an interaction (such as a questionnaire) with a user device. A user device may include computing device 104, a “smartphone,” cellular mobile phone, desktop computer, laptop, tablet computer, internet-of-things (IOT) device, wearable device, among other devices. User device may include any device that is capable for communicating with computing device 104, database, or able to receive, transmit, and/or display nutrient profile 112, nourishment consumption program 120, compatible alimentary elements, etc., for instance via a data network technology such as 3G, 4G/LTE, 5G, Wi-Fi (IEEE 802.11 family standards), and the like. User device may include devices that communicate using other mobile communication technologies, or any combination thereof, for short-range wireless communication (for instance, using Bluetooth and/or Bluetooth LE standards, AirDrop, Wi-Fi, NFC, etc.), and the like.

Continuing in reference to FIG. 1, providing a representation of the nourishment consumption program 120 may include providing an audiovisual notification. An “audiovisual notification,” as used in this disclosure, is an audio and/or visual based notification that may be displayed via an interface with computing device 104. An audiovisual notification may include a prompt to order an alimentary element. An audiovisual notification may include a compatible alimentary element from an alimentary element program. Providing a representation of the nourishment consumption program 120 may include linking, for instance, a subject's calendar application with an alimentary element program. In non-limiting illustrative examples, a user device may be configured to set timed reminders for subject to consume foods, where foods are determined as a function of current location, options, etc.

Continuing in reference to FIG. 1, after providing the nourishment consumption program 120, computing device 104 may update the nutrient profile 112 as a function of subject nutrient consumption. Defined intervals for updating nutrient profile 112 may be set by computing device 104 using reactive computing. “Reactive computing,” as used in this disclosure is a declarative programming paradigm that is concerned with data streams and the propagation of change in such data over sampled time period. Reactive computing may also be referred to as “reactive programming.” Reactive computing may be used to iteratively sample data inputs and, according to an internal “clock”, generate iterative outputs in real-time, as the input data is collected. As used in this disclosure, input data may include nutritional input and/or physiological data 108, for instance as input by subject or collected by a physiological sensor, respectively, and received by computing device 104. As used in this disclosure, output data may include nutrient profile 112 and nourishment consumption program 120. Input data may include data that is generated as training data, and outputs may be from a machine-learning process. Computing device 104 may be configured, using reactive computing, to express static (such as arrays) and/or dynamic (such as event emitters) “data streams” with relative ease, and also communicate an inferred dependence within the associated “execution model” which facilitate the automatic propagation of the changed data flow. For instance, computing device 104 may be configured to employ a trained machine-learning process or model, which describes a mathematical relationship between a particular input to a particular output as the “execution model” to automatically propagate outputs form the incoming signal data. Essentially, computing device 104 may use reactive computing to iteratively receive nutritional inputs and/or physiological data 108 (inputs) and generate nutrient profile 112 and nourishment consumption program 120 (outputs) at regular scheduled intervals, including as data is received (real-time), according to trained machine-learning models such as the nutrient machine-learning model 116. Computing device 104 may use the nutrient profile 112 and an alimentary element program stored in database to generate nourishment consumption program 120 each time nutrient profile 112 is updated throughout the subject's day.

Continuing in reference to FIG. 1, reactive computing may include “model-view-controller” (MVC) architecture, wherein reactive programming may facilitate changes in an underlying model that are reflected automatically in an associated view. For instance, a trained nutrient machine-learning model 116, which may correlate physiological data 108 to nutrient profile 112 data, wherein the nutrient profile 112 may include numerical values for each nutrient. Reactive computing may be performed by computing device 104 using reactive extensions, such as RxJs, RxJAva, RxPy, RxSwift, and other APIs. Reactive computing may be implemented using any type of change propagation algorithm, such as a pull, push, and/or push-pull type approach to data propagation. Reactive computing may be any object-oriented reactive programming (OORP), functional reactive programming (FRP), or the like. Reactive programming may be implemented using for instance rule-based reactive programming languages such as through using relation algebra with Ampersand, Elm, and/or Observable. Persons skilled in the art, upon review of this disclosure in its entirety, will be aware of the various ways in which to implement reactive computing to sample inputs and provide updates in real-time, or at any defined interval.

Still referring to FIG. 1, an audiovisual notification may include hydration notification alerting a user to increase, maintain or decrease their intake of fluids based on an update the nutrient profile 112 as a function of subject nutrient consumption. In some embodiments, a hydration notification may consist of an audiovisual notification transmitted to a user device such as a smart water bottle configured to alert a user when that need to hydrate. A “water bottle,” for the purposes of this disclosure, is a container that is configured to contain or hold a liquid in a portable manner. A “smart device,” for the purposes of this disclosure, is a device that is communicatively connected to one or more other devices using a wireless protocol and is able to operate interactively with those other devices. A “smart water bottle,” for the purposes of this disclosure, is a smart device that is configured to function as a water bottle. A smart water bottle may be crafted from durable, BPA-free materials, and be equipped with customizable alerts through gentle vibrations or LED notifications on the bottle, ensuring that users stay on track with their hydration targets. The device's LED display or light indicators may offer a quick glance at the user's hydration status, with green indicating sufficient hydration and yellow or red signaling the need for more water intake. Moreover, the smart water bottle can seamlessly integrate with wearable devices like fitness trackers, providing a holistic view of the user's health.

Still referring to FIG. 1, in some embodiments audiovisual notifications may include notification of encouragement to a user. A “notification of encouragement,” for the purposes of this disclosure, is a notification that prompts or seeks to cause a user to perform a task. For example, a user undergoing intermittent fasting may receive alerts encouraging the user to continue their fast by providing medical benefits that occur as a result of a fasting period. In some embodiments, “tasks” may be identified from nourishment program 120. For example, each step, in some embodiments, of nourishment program 120 may be identified as a step. In some embodiments, consumption patterns or consumption schedules may be considered to be tasks. As a non-limiting example, if the consumption pattern is 1 small meal then 1 large meal then one small meal, a plurality of tasks may be generated, with each task relating to the consumption of a meal.

Still referring to FIG. 1, In some embodiments, content for the notification of encouragement may be retrieved from a lookup table (LUT). A “lookup table,” for the purposes of this disclosure, is an array of data that maps input values to output values. The lookup table may be used to replace a runtime computation with an array indexing operation. As a non-limiting example, LUT may contain tasks correlated to encouragement for those tasks. For example, a task of “drink 2 liters of water today” may be correlated to an encouragement of “staying hydrated helps lessen the burden on your kidneys and liver.” In some embodiments, computing device may retrieve content for the notification of encouragement from the LUT, then insert the content into the notification.

With continued reference to FIG. 1, the medical benefits or encouragement may be received from a database as disclosed in this disclosure or by using a web crawler. A “web crawler,” as used herein, is a program that systematically browses the internet for the purpose of Web indexing. The web crawler may be seeded with platform URLs, wherein the crawler may then visit the next related URL, retrieve the content, index the content, and/or measures the relevance of the content to the topic of interest. Computing device 104 may train a web crawler to browse health and medical related sites based on the nutritional needs and types of diet a user is undergoing. As a non-limiting example, web crawler may be configured to browse nih.gov, mayoclinic.org or other such health sites. Such information may be indexed by the web crawler into a database for generating the notification. In some embodiments, web crawler may locate information as a function of keywords. For example, keywords may include a stage of fasting, a type of diet, an eating pattern, a hydration pattern, and the like.

Still referring to FIG. 1, computing device may generate notification of encouragement using generative AI. In some embodiments, generative AI may include a large language model (LLM). A “large language model,” as used herein, is a deep learning algorithm that can recognize, summarize, translate, predict and/or generate text and other content based on knowledge gained from massive datasets. Large language model may be trained on large sets of data; for example, training sets may include greater than 1 million words. Training sets may be drawn from diverse sets of data such as, as non-limiting examples, novels, blog posts, articles, emails, and the like. In some embodiments, training sets may include a variety of subject matters, such as, as nonlimiting examples, medical tests, romantic ballads, beat poetry, emails, advertising documents, newspaper articles, and the like. In some embodiments, training sets may be drawn from health blogs, cooking blogs, fitness advice websites, and the like.

With continued reference to FIG. 1, in some embodiments, LLM may be generally trained. For the purposes of this disclosure, “generally trained” means that LLM is trained on a general training set comprising a variety of subject matters, data sets, and fields. In some embodiments, LLM may be initially generally trained. In some embodiments, for the purposes of this disclosure, LLM may be specifically trained. For the purposes of this disclosure, “specifically trained” means that LLM is trained on a specific training set, wherein the specific training set includes data including specific correlations for LLM to learn. As a non-limiting example, LLM may be generally trained on a general training set, then specifically trained on a specific training set. As a non-limiting example, specific training set may include textual works 108. As a non-limiting example, specific training set may include scholastic works. As a non-limiting example, specific training set may include information from nourishment program database 604. In some embodiments, specific training set may be received from information collected by the web crawlers as disclosed above.

With continued reference to FIG. 1, LLM, in some embodiments, may include Generative Pretrained Transformer (GPT), GPT-2, GPT-3, GPT-4, and the like. GPT, GPT-2, GPT-3, and GPT-4 are products of Open AI Inc., of San Francisco, CA. LLM may include a text prediction based algorithm configured to receive an article and apply a probability distribution to the words already typed in a sentence to work out the most likely word to come next in augmented articles. For example, if the words already typed are “Nice to meet”, then it is highly likely that the word “you” will come next. LLM may output such predictions by ranking words by likelihood or a prompt parameter. For the example given above, the LLM may score “you” as the most likely, “your” as the next most likely, “his” or “her” next, and the like. LLM may include an encoder component and a decoder component.

Still referring to FIG. 1, LLM may include a transformer architecture. In some embodiments, encoder component of LLM may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 1, LLM and/or transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation.

With continued reference to FIG. 1, an attention mechanism may represent an improvement over a limitation of the Encoder-Decoder model. The encoder-decider model encodes the input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, LLM may predict the next word by searching for a set of position in a source sentence where the most relevant information is concentrated. LLM may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

Still referring to FIG. 1, an attention mechanism may include generalized attention self-attention, multi-head attention, additive attention, global attention, and the like. In generalized attention, when a sequence of words or an image is fed to LLM, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, LLM may pick up particular parts at different positions in the input sequence and over time compute an initial composition of the output sequence. In multi-head attention, LLM may include a transformer model of an attention mechanism. Attention mechanisms, as described above, may provide context for any position in the input sequence. For example, if the input data is a natural language sentence, the transformer does not have to process one word at a time. In multi-head attention, computations by LLM may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bahdanau attention mechanism), LLM may make use of attention alignment scores based on a number of factors. These alignment scores may be calculated at different points in a neural network. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, LLM may either attend to all source words or predict the target sentence, thereby attending to a smaller subset of words.

With continued reference to FIG. 1, multi-headed attention in encoder may apply a specific attention mechanism called self-attention. Self-attention allows the models to associate each word in the input, to other words. So, as a non-limiting example, the LLM may learn to associate the word “you”, with “how” and “are”. It's also possible that LLM learns that words structured in this pattern are typically a question and to respond appropriately. In some embodiments, to achieve self-attention, input may be fed into three distinct fully connected layers to create query, key, and value vectors. The query, key, and value vectors may be fed through a linear layer; then, the query and key vectors may be multiplies using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

With continued reference to FIG. 1, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 1, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

With continued reference to FIG. 1, transformer architecture may include a decoder. Decoder may a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With continued reference to FIG. 1, in some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 1, first multi-headed attention layer may be configured to not condition to future tokens. As a non-limiting example, when computing attention scores on the word “am”, decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “Os” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.”

With continued reference to FIG. 1, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 1, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

With continued reference to FIG. 1, decoder may take this output and add it to the decoder inputs. Decoder may continue decoding until a token is predicted. Decoder may stop decoding once it predicts an end token.

With continued reference to FIG. 1, in some embodiment, decoder may be stacked N layers high, with each layer taking in inputs from the encoder and layers before it. Stacking layers may allow LLM to learn to extract and focus on different combinations of attention from its attention heads.

With continued reference to FIG. 1, LLM may receive an input. Input may include a string of one or more characters. For example, input may include one or more words, a sentence, a paragraph, a thought, a query, and the like. In some embodiments, input may be received from a user device. In some embodiments, input may include a task, a task completion level, a nourishment consumption program 120, a consumption pattern, a consumption schedule, and the like.

With continued reference to FIG. 1, LLM may generate output. In some embodiments, LLM may include multiple sets of transformer architecture as described above. Output may include a textual output. A “textual output,” for the purposes of this disclosure is an output comprising a string of one or more characters. Textual output may include for example, an encouragement notification or text for the encouragement notification. In some embodiments, encouragement notification may include information from the LUT or data crawler above that has been rewritten to be more encouraging or fit a particular user in some way.

With continued reference to FIG. 1, LLM may be further trained using user feedback. User feedback may be received from user device. User feedback may include explicit feedback such as “5 starts” “great” “like,” and the like. In some embodiments, user feedback may be implicit. Implicit user feedback may be inferred from user actions. For example, if a certain amount of time passes before a user completes the task associated with the notification of encouragement, then processor may identify that as negative feedback. For example, if notification of encouragement results in completion of a task, then processor may determine this to be positive feedback.

Still referring to FIG. 1, computing device may be configured to pair a user to a nutritional expert as a function of the updated nutrient profile 112. For example, when data shows a user is consistently not hitting their fasting/hydrating/consumption pattern goals, computing device may match a user to a nutritional expert based on the nutrient profile 112, physiological data 108. Classifying the user to an expert may include using an expert machine-learning model wherein the training data includes data correlating elements of a nutrient profile and physiological data to a plurality of nutritional expert profiles. A nutritional expert profile may be a data structure containing the credentials, expertise, contact information, and the like of the nutritional expert. A nutritional expert matched to the user may be displayed through a user interface as described above.

Still referring to FIG. 1, computing device may select one or more nutritional experts as a function of the nourishment completion program 120. In some embodiments, nutritional expert may be retrieved from an expert database. Expert database may be populated with a plurality of nutritional experts associated with one or more factors. Factors may be specialties, skills, location, and the like of the nutritional expert. In some embodiments, only certain experts may be associated with certain types of nourishment consumption program 120. Computing device may be configured to retrieve the one or more nutritional experts associated with a nourishment consumption program 120 and select a nutritional expert for the user. In some embodiments, nutritional expert may be selected as a function of geographic location of the nutritional expert. For example, in some cases, only nutritional experts within a set radius of the user may be considered for selection.

Still referring to FIG. 1, audiovisual notification may include achievement badges for every task of a consumption/hydration/fasting pattern that a user accomplishes in regard to nourishment consumption program 120. A digital achievement badge is a virtual recognition symbol awarded to individuals for accomplishing specific tasks, milestones, or achievements within a digital or online platform. These badges serve as a visual representation of a person's accomplishments and can be prominently displayed on their profile or within the platform. Achievement badges are designed with graphics or icons that symbolize the nature of the achievement. They may vary in complexity and may include elements that reflect the difficulty level or significance of the accomplishment. These badges may come with a brief description or title that provides context for the achievement.

Still referring to FIG. 1, displaying nourishment consumption program 120 may include displaying a hydration meter. A “hydration meter,” as used herein, in visual indicator of a hydration level. A hydration level may include linguistic variables such as dehydrated to hydrated or may be represented as a percentage. In some embodiments, computing device 104 may receive a type of beverage consumed by the user and indicate the hydration level offered by the beverage through the hydration meter. For example, the hydration meter may be programed to scale out beverages consumed based on a daily intake value. A can of soda may be depicted to offer a low level of hydration while a sport drink may be depicted to offer a high level of hydration. In some embodiments, hydration level may be determined from nutrient quantifier 128. For example, nutrient quantifier may quantify the hydrating properties (positive of negative) of a nutrient. In some embodiments, the hydration level for hydration meter to display may be calculated by summing nutrient quantifiers 128 for every nutrient consumed by a user (or scheduled to be consumed by a user) within a lookback timeframe. Lookback timeframe may be anywhere from 10 minutes to 2 days. Lookback timeframe may be 1 hour. Lookback timeframe may be 3 hours. Lookback timeframe may be 12 hours. Lookback timeframe may be 3 hours. Lookback timeframe may be 1 day. Lookback timeframe may be 2 days. In some embodiments, hydration meter may include a dial component. A “dial component,” for the purposes of this disclosure is a radial visual feature that displays a range of possible values for an indicator. In some embodiments, hydration meter may include an arrow overlayed on a dial component, wherein the arrow indicates the hydration level.

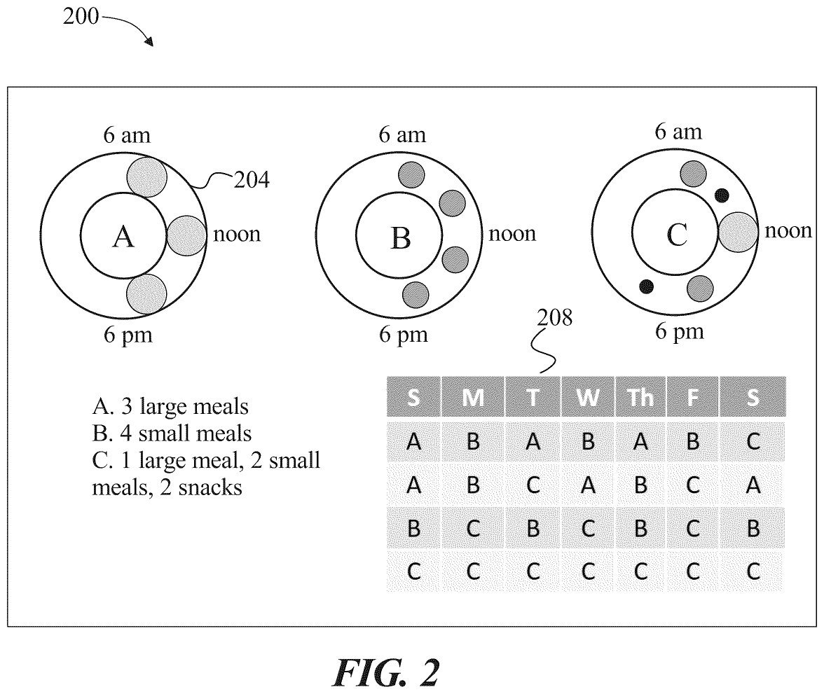

Referring now to FIG. 2, a non-limiting exemplary embodiment of nourishment consumption program 120 is illustrated. Nourishment consumption program 120 may include several consumption patterns 204, for instance as designated ‘A’, ‘B’, and ‘C’. Each may include the timing and designation of meals, snacks, caloric content, etc. Consumption patterns 204 may include daily patterns of nourishment consumption. Consumption patterns may be organized into consumption schedule 208, such as a monthly schedule. Nourishment consumption program 120 may include a variety of consumption patterns 204 organized into consumption schedules 208, for instance based on physiological goals such as lowering BMI, fighting obesity, ameliorating a particular disease (type-2 diabetes), or addressing a symptom (improving sleep deprivation). Nourishment consumption program 120 may include a variety of consumption patterns 204 organized into consumption schedules 208 with particular identified alimentary elements, such as particular grains, meats, fruits, diary, vegetables, and the like, arranged in dietary paradigms such as ‘ketogenic diet’, ‘low glycemic index diet’, ‘plant-based diet’, etc., where the timing of nourishment is guided toward a goal. Such a goal may include maintaining a certain level of iron in the body or keeping cholesterol or blood sugar within a particular range.

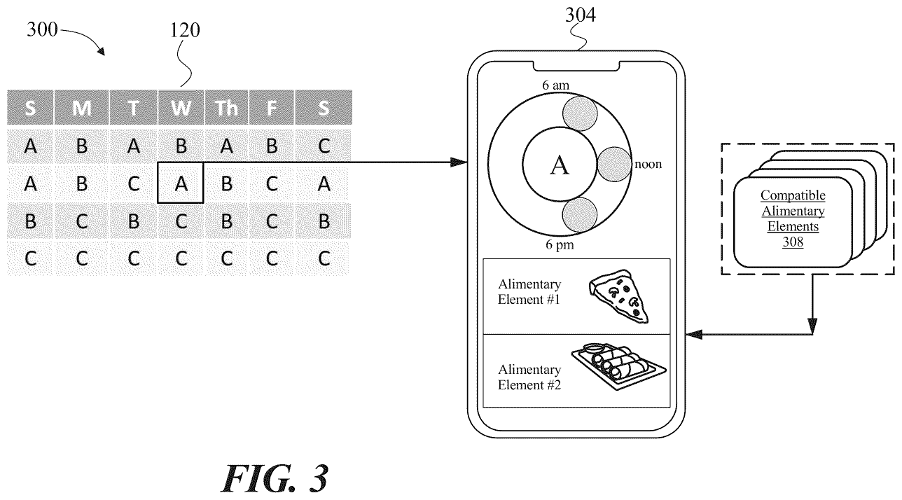

Referring now to FIG. 3, a non-limiting exemplary embodiment of nourishment consumption program 120 provided on a user device is illustrated. User device 304 may include computing device 104, a “smartphone,” cellular mobile phone, desktop computer, laptop, tablet computer, internet-of-things (IOT) device, wearable device, among other devices. User device may include any device that is capable for communicating with computing device 104, database, or able to receive, transmit, and/or display nutrient profile 112, nourishment consumption program 120, compatible alimentary elements 308. User device 304 may provide a nutrient profile 112, for instance as a collection of metrics determined from physiological data 108 data. User device may provide data concerning average levels of nutrients, nutrient lows, nutrient highs, etc. User device may link timing of foods to preemptive ordering interface for ordering an alimentary element, for instance through a designated mobile application, mapping tool or application, etc. User device may link nourishment consumption program 120 to a scheduling application, such as a ‘calendar’ feature on user device, which may set timers, alarms, and the like.

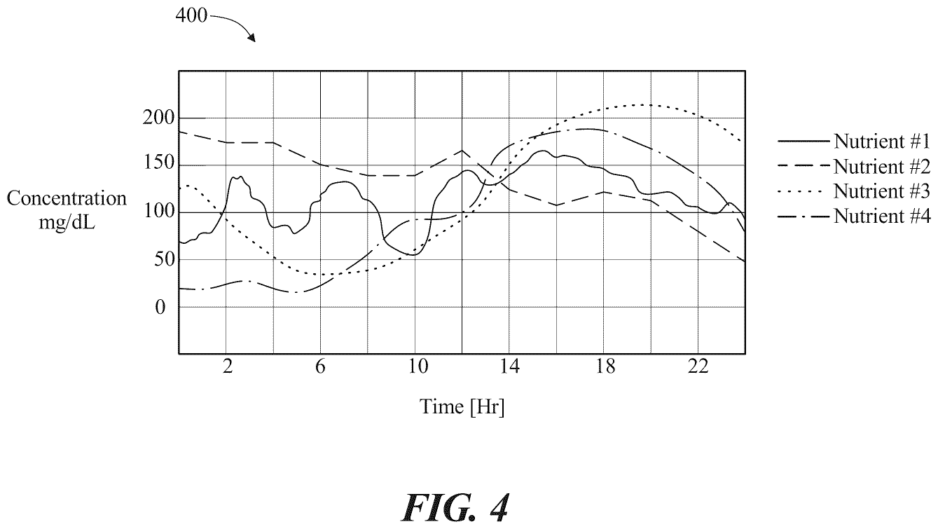

Referring now to FIG. 4, a non-limiting illustrative embodiment of nutrient profile 112 data is illustrated. Physiological data 108 may include data such as blood concentration of nutrients (mg/dL). Computing device 104 may receive physiological data 108, including nutritional input, such as times of meals consumed, and nutrition facts of alimentary elements consumed. Nutrient machine-learning process 116 may determine relationships between consumption of particular alimentary elements and the concentration of nutrient in a physiological data. For instance, as depicted in FIG. 4, nutrient #1 may correspond to blood sugar, which generally oscillates between 50 and 140 mg/dL throughout the day for healthy adults. From time point ‘0 hour’, or 8 am for when subject awakens to approximately ‘+10 hours’ blood sugar remains in normal nutrient threshold values. At approximately, ‘+10 hours’ time, the subject consumes the largest meal of the day, which is diner at approximately 6 pm, and then consumes a nutrient-rich alimentary element approximately 1.5 hours later prior to bed leading to blood sugar between 140 and 199 mg/dL, implying prediabetes without optimal meals and timing. A plurality of nutrients may reach local maxima nutrient amounts during digestion in the evening-night (14-18 hours) if the largest amount of nutrients are consumed during the last few hours awake. Such a pattern may suggest eating smaller meals or spreading nutrients out over a longer time. Inflection points in the function may match to timing of meals. The period after the infection (+1 hour post meal, +2 hours, etc.) may be used to train a machine-learning model to determine rates associated with absorption and metabolism with that subject. Persons skilled in the art may appreciate that performed over sufficiently large periods, and with a large variety of alimentary elements, for a large set of nutrients, nutrient machine-learning model 116 may accurately determine nutrient profile 112 metrics, parameters, numerical values, etc.

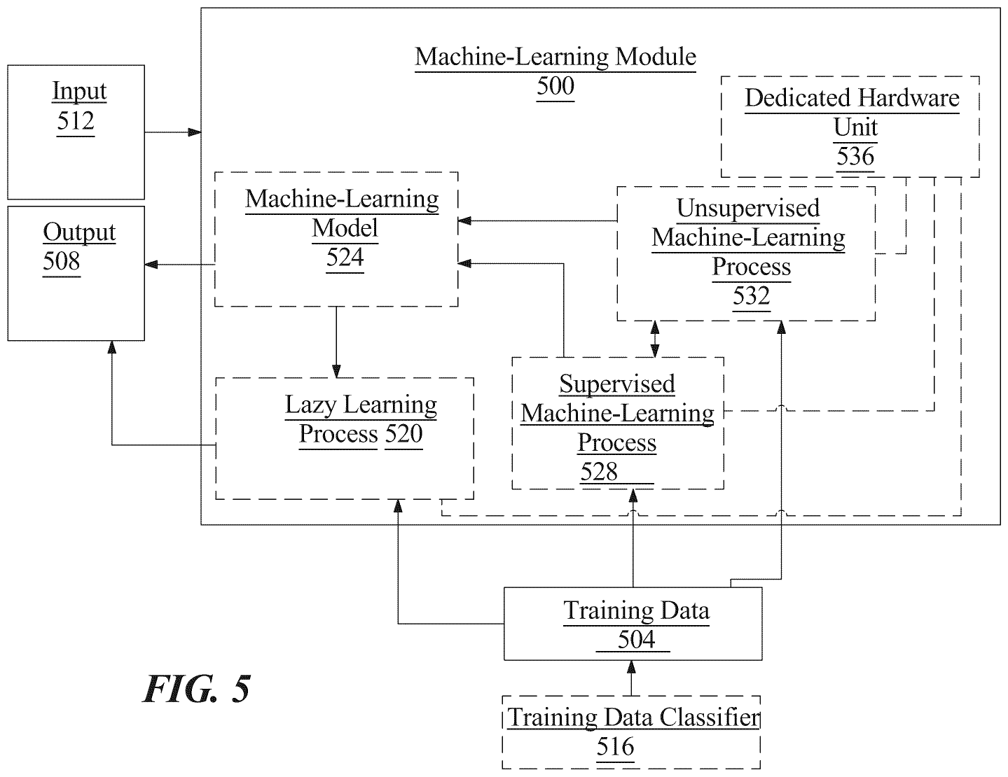

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm instantiated in hardware or software logic, data structures, and/or functions that will be performed by a computing device/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative examples, training data may include nutrients correlated to expected nutrient levels or examples of consumption of a nutrient correlated to presence of a nutrient in physiological data.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to age groups, genders, metabolism types, lifestyles, and the like.

Still referring to FIG. 5, computing device 504 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 504 may then calculate a likelihood table by calculating probabilities of different data entries and classification labels. Computing device 504 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 5, computing device 504 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 5, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿαᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 5, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 5, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 5, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 5, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 5, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 5, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 5, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 5, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:X_{new}=X−Xₘᵢₙ/Xₘₐₓ−Xₘᵢₙ. Feature scaling may include mean normalization, which involves use of a mean value of a set and/or subset of values, Xₘₑₐₙ with maximum and minimum values: X_{new}=X−Xₘₑₐₙ/Xₘₐₓ−Xₘᵢₙ. Feature scaling may include standardization, where a difference between X and Xₘₑₐₙ is divided by a standard deviation σ of a set or subset of values: X_{new}=X−Xₘₑₐₙ/σ. Scaling may be performed using a median value of a set or subset X_{median} and/or interquartile range (IQR), which represents the difference between the 25^{th} percentile value and the 50^{th} percentile value (or closest values thereto by a rounding protocol), such as: X_{new}=X−X_{median}/IQR. Persons skilled in the art, upon reviewing the entirety of this disclosure, will be aware of various alternative or additional approaches that may be used for feature scaling.

Further referring to FIG. 5, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs as described in this disclosure as inputs, outputs as described in this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

With further reference to FIG. 5, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 5, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 532 may not require a response variable; unsupervised processes 532 may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 5, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 5, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 5, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 5, one or more processes or algorithms described above may be performed by at least a dedicated hardware unit 536. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 536 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 536 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 536 to perform one or more operations described herein, such as evaluation of model and/or algorithm outputs, one-time or iterative updates to parameters, coefficients, weights, and/or biases, and/or any other operations such as vector and/or matrix operations as described in this disclosure.

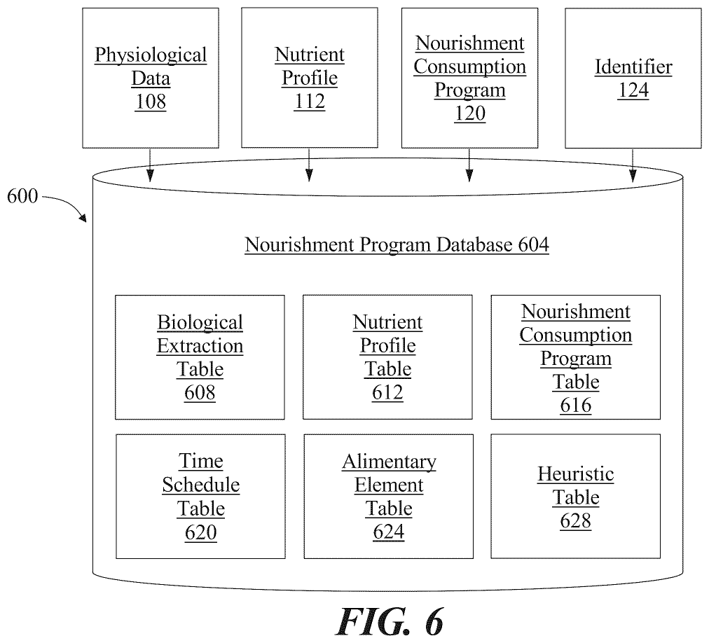

Referring now to FIG. 6, a non-limiting exemplary embodiment 600 of a nourishment program database 604 is illustrated. Physiological data 108 for a plurality of subjects, for instance for generating a training data classifier 516, may be stored and/or retrieved in nourishment program database 604. Physiological data 108 data from a plurality of subjects for generating training data 504 may also be stored and/or retrieved from a nourishment program database 604. Computing device 104 may receive, store, and/or retrieve training data 504, wearable device data, physiological sensor data, and the like, from nourishment program database 604. Computing device 104 may store and/or retrieve nutrient machine-learning model 116, among other determinations, I/O data, models, and the like, in nourishment program database 604.

Continuing in reference to FIG. 6, nourishment program database 604 may be implemented, without limitation, as a relational database, a key-value retrieval database such as a NOSQL database, or any other format or structure for use as a database that a person skilled in the art would recognize as suitable upon review of the entirety of this disclosure. Nourishment program database 604 may alternatively or additionally be implemented using a distributed data storage protocol and/or data structure, such as a distributed hash table and the like. Nourishment program database 604 may include a plurality of data entries and/or records, as described above. Data entries in a nourishment program database 604 may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistent with this disclosure.

Further referring to FIG. 6, nourishment program database 604 may include, without limitation, physiological data table 608, nutrient profile table 612, nourishment consumption program table 616, time schedule table 620, alimentary element table 624, and/or heuristic table 628. Determinations by a machine-learning process, machine-learning model, ranking function, and/or classifier, may also be stored and/or retrieved from the nourishment program database 604. As a non-limiting example, nourishment program database 604 may organize data according to one or more instruction tables. One or more nourishment program database 604 tables may be linked to one another by, for instance in a non-limiting example, common column values. For instance, a common column between two tables of nourishment program database 604 may include an identifier of a submission, such as a form entry, textual submission, accessory device tokens, local access addresses, metrics, and the like, for instance as defined herein; as a result, a search by a computing device 104 may be able to retrieve all rows from any table pertaining to a given submission or set thereof. Other columns may include any other category usable for organization or subdivision of data, including types of data, names and/or identifiers of individuals submitting the data, times of submission, and the like; persons skilled in the art, upon reviewing the entirety of this disclosure, will be aware of various ways in which data from one or more tables may be linked and/or related to data in one or more other tables.

Continuing in reference to FIG. 6, in a non-limiting embodiment, one or more tables of an nourishment program database 604 may include, as a non-limiting example, a physiological data table 608, which may include categorized identifying data, as described above, including genetic data, epigenetic data, microbiome data, physiological data, and the like. Physiological data table 608 may include physiological data 108 categories according to metabolism, absorption, etc., categories, and may include linked tables to mathematical expressions that describe the impact of each physiological data 108 datum on nutrient profile 112, for instance threshold values for gene expression, etc., as it relates to nutrient levels. One or more tables may include nutrient profile table 612, which may include data regarding physiological data 108, thresholds, values, categorizations, and the like, that system 100 may use to calculate, derive, filter, retrieve and/or store current nutrient levels, metabolic rates, absorption rates, digestive difficulties, and the like. One or more tables may include nourishment consumption program table 616, which may include data regarding times to eat, identifiers of alimentary elements, schedules, diet types, and the like. Nourishment consumption program table 616 may include data from alike subjects with similar physiological data 108, and the like, that system 100 may use to calculate, derive, filter, retrieve and/or store meal times, for instance timing blood sugar based on meals as a function of alike subjects' meal scheduling. One or more tables may include time schedule table 620, which may include data including times of previous consumption, future scheduled consumption, and the like, that system 100 may use to link to nutrient profile 112 and/or nourishment consumption program 120. One of more tables may include an alimentary element table 624, which may include identifiers and times associated with alimentary elements. One or more tables may include, without limitation, a heuristic table 628, which may organize rankings, scores, models, outcomes, functions, numerical values, scales, arrays, matrices, and the like, that represent determinations, probabilities, metrics, parameters, values, and the like, include one or more inputs describing potential mathematical relationships, as described herein.

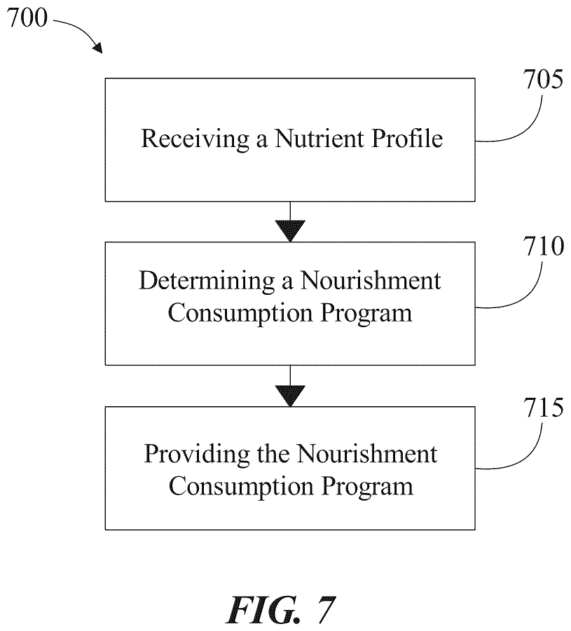

Referring now to FIG. 7, an exemplary embodiment 700 of a method for timing impact of nourishment consumption is illustrated. At step 705, computing device 104 is configured for a nutrient profile 112 of a subject, wherein the nutrient profile 112 maps physiological data of the subject to current nutrient levels of the subject. Receiving the nutrient profile 112 may include training a nutrient machine-learning model 116 with training data that includes a plurality of data entries wherein each entry correlates physiological data 108 to current nutrient levels of the subject, and determining the nutrient profile as a function of the nutrient machine-learning model; this may be implemented, without limitation, as described above in FIGS. 1-6.

Still referring to FIG. 7, at step 710, computing device 104 is configured for determining, using the nutrient profile 112, a nourishment consumption program 120, wherein the nourishment consumption program includes at least an alimentary element, and a time of day for consuming the alimentary element wherein the time of day is determined as a function of the nutrient profile 112 and the current nutrient level of the subject. Determining the nourishment consumption program 120 may include retrieving an alimentary element program comprising compatible alimentary elements. Determining the nourishment consumption program 120 may include identifying a compatible alimentary element to address a datum of the nutrient profile 112. Computing device 104 may calculate a change in the nutrient profile 112 as a function of a time of day for consuming the compatible alimentary element. Nourishment consumption program 120 may include a queue of a plurality of compatible alimentary elements, wherein each compatible alimentary element includes an identifier. Nourishment consumption program 120 may include the time of day associated with the identifier, wherein the time of day is selected based on the nutrient profile 112. Nourishment consumption program 120 may include a nutrient quantifier for adjusting the nutrient profile 112 as a function of consumption of an alimentary element associated with the identifier; this may be implemented, without limitation, as described above in FIGS. 1-6.

Continuing in reference to FIG. 7, at step 715, computing device 104 is configured for providing, to the subject, the nourishment consumption program 120. Providing the nourishment consumption program 120 may include generating, via a graphical user interface, a representation of the nourishment consumption program 120. Providing the nourishment consumption program 120 may include updating the nutrient profile 112 as a function of subject nutrient consumption; this may be implemented, without limitation, as described above in FIGS. 1-6.

Continuing in reference to FIG. 7, at step 720, computing device 104 is configured for providing, to the subject, the nourishment consumption program 120. Providing the nourishment consumption program 120 may include generating, via a graphical user interface, a representation of the nourishment consumption program 120. After providing the nourishment consumption program, computing device 104 may update the nutrient profile 112 as a function of subject nutrient consumption; this may be implemented, without limitation, as described above in FIGS. 1-6.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

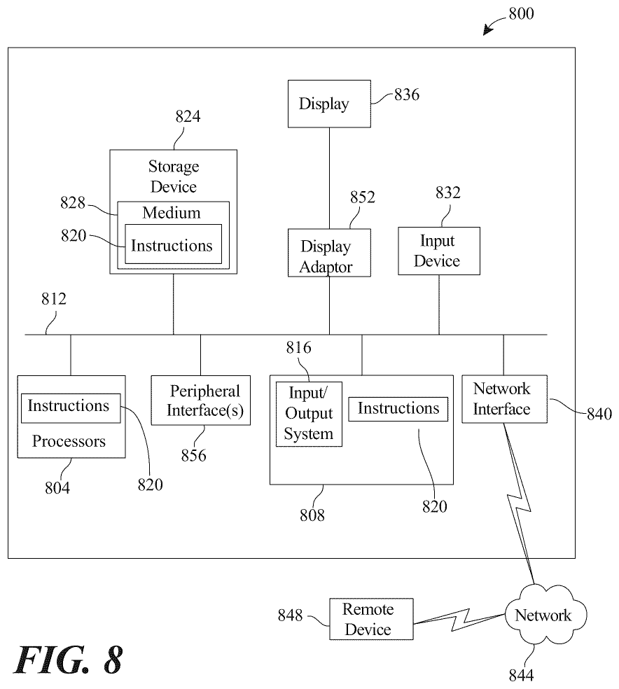

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC)

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A system for timing impact of nourishment consumption, the system comprising:
a computing device, the computing device configured to:
receive training data comprising physiological data, correlated to current nutrient levels of a subject;
train a nutrient machine-learning model using the training data;
generate a nutrient profile of the subject utilizing the nutrient machine-learning model;
determine, using the nutrient profile, a nourishment consumption program;
provide, to the subject, the nourishment consumption program;
receive a set of nutrition consumption data of the subject as a function of the nourishment consumption program;
generate an updated nutrient profile as a function of the set of nutrition consumption data and the defined time intervals; and
generate one or more audiovisual notifications based on the nutrient consumption program, wherein the one or more audiovisual notifications comprises a notification of encouragement;
provide, to the subject, an updated consumption pattern of the nourishment consumption program as a function of the set of nutrition consumption data and the updated nutrient profile at each defined time interval.

2. The system of claim 1, wherein physiological data comprises food intolerances of a subject.

3. The system of claim 1, wherein the nutrient machine-learning model is configured to:
receive a nutritional input;
determine relationships between consumption of particular alimentary elements and the concentration of nutrients in physiological data; and
output the nutrient profile comprising a nutritional deficiency.

4. The system of claim 1, wherein the nourishment consumption program includes:
at least an alimentary element; and
a consumption pattern for consuming the at least an alimentary element, wherein:
the consumption pattern includes a time of day; and
the time of day is determined as a function of the nutrient profile and the current nutrient levels of the subject.

5. The system of claim 1, wherein providing the nourishment consumption program further comprises:
providing a representation of the nourishment consumption program to a user device of the subject in the form of an audiovisual notification;
linking the nourishment consumption program to a calendar application of the user device of the subject; and
setting timed reminders on the user device of the subject to consume at least one alimentary element as a function of a current location of the subject.

6. The system of claim 1, wherein the at least an audiovisual notification comprises a hydration notification.

7. The system of claim 6, wherein the computing device is further configured to transmit a hydration notification to a smart water bottle.

8. The system of claim 1, wherein the audiovisual notification comprises medical benefits associated with the nutrient consumption program.

9. The system of claim 1, wherein the audiovisual notification comprises an achievement badge.

10. The system of claim 1, wherein the computing device further configured to classify the subject to a nutrient expert using an expert machine learning model.

11. A method for timing impact of nourishment consumption, the method comprising:
receiving, by a computing device, training data comprising physiological data, correlated to current nutrient levels of a subject;
training, by the computing device, a nutrient machine-learning model using the training data;
generating, by the computing device, a nutrient profile of the subject utilizing the nutrient machine-learning model;
determining, by the computing device, using the nutrient profile, a nourishment consumption program;
providing, by the computing device, to the subject, the nourishment consumption program;
receiving, by the computing device, a set of nutrition consumption data of the subject as a function of the nourishment consumption program;
generating, by the computing device, an updated nutrient profile as a function of the set of nutrition consumption data and the defined time intervals;
generating, by the computing device, one or more audiovisual notifications based on the nutrient consumption program, wherein the one or more audiovisual notifications comprises a notification of encouragement;
providing, by the computing device, to the subject, an updated consumption pattern of the nourishment consumption program as a function of the set of nutrition consumption data and the updated nutrient profile at each defined time interval.

12. The method of claim 11, wherein physiological data comprises food intolerances of a subject.

13. The method of claim 11, wherein the nutrient machine-learning model is configured to:
receive a nutritional input;
determine relationships between consumption of particular alimentary elements and the concentration of nutrients in physiological data; and
output the nutrient profile comprising a nutritional deficiency.

14. The method of claim 11, wherein the nourishment consumption program includes:
at least an alimentary element; and
a consumption pattern for consuming the at least an alimentary element, wherein:
the consumption pattern includes a time of day; and
the time of day is determined as a function of the nutrient profile and the current nutrient levels of the subject.

15. The method of claim 11, wherein providing the nourishment consumption program further comprises:
providing a representation of the nourishment consumption program to a user device of the subject in the form of an audiovisual notification;
linking the nourishment consumption program to a calendar application of the user device of the subject; and
setting timed reminders on the user device of the subject to consume at least one alimentary element as a function of a current location of the subject.

16. The method of claim 11, wherein the at least an audiovisual notification comprises a hydration notification.

17. The method of claim 16, further comprising transmitting, by the computing device, the hydration notification to a smart water bottle.

18. The method of claim 11, wherein the audiovisual notification comprises medical benefits associated with the nutrient consumption program.

19. The method of claim 11, wherein the audiovisual notification comprises an achievement badge.

20. The method of claim 11, wherein generating the updated nutrient profile further comprises classifying the subject to a nutrient expert using an expert machine learning model.
